(19) United States
(12) Patent Application Publication
(10) Pub. No.: US 2003/0018311 A1
(43) Pub. Date: Jan. 23, 2003

(54) Unitary absorbent layer

(75) Inventors: Peter A. Graef, Puyallup, WA (US); Colin Elston, Gig Harbor, WA (US); Daniel T. Bunker, Karhula (FI); Fred B. Howard, Olalla, WA (US); Jeffrey D. Mathews, Puyallup, WA (US); Shahrokh A. Naieni, Seattle, WA (US)

Correspondence Address: WEYERHAEUSER COMPANY, INTELLECTUAL PROPERTY DEPT., CH 1J27, P.O. BOX 9777, FEDERAL WAY, WA, 98063, US

(73) Assignee: Weyerhaeuser Company

(21) Appl. No.: 10/184,339

(22) Filed: Jun. 26, 2002

Related U.S. Application Data
(63) Continuation of application No. 09/137,453, filed on Aug. 20, 1998.
Application No. 09/137,453 is a continuation of application No. PCT/US97/22341, filed on Dec. 5, 1997.
(60) Provisional application No. 60/032,794, filed on Dec. 6, 1996.

(51) Int. Cl. A61F 13/15; A61F 13/20

(52) U.S. Cl. 604/374; 604/366; 604/368; 604/372; 604/378

(57) ABSTRACT

A unitary absorbent layer composed of a crosslinked cellulosic fibers and a binder is disclosed. In a preferred embodiment, the binder is a bicomponent binding fiber. In combination with one or more other layers in an absorbent article, the unitary absorbent layer can rapidly acquire, distribute, temporarily store, and then release the acquired liquid to other liquid retention layers. Methods for forming the unitary absorbent layer are also disclosed.

### CROSS-REFERENCE TO RELATED APPLICATIONS

This application is a continuation of copending international application number PCT/US97/22341, filed Dec. 5, 1997, which is a continuation-in-part of U.S. patent application Ser. No. 60/032,794, filed Dec. 6, 1996, priority of the filing dates of which is hereby claimed under 35 U.S.C. §§ 120 and 119, respectively. Each of these applications is incorporated herein by reference.

### FIELD OF THE INVENTION

The present invention relates to an absorbent layer and methods for making the same and, more particularly, to a unitary absorbent layer.

### BACKGROUND OF THE INVENTION

Cellulose fibers derived from wood pulp are used in a variety of absorbent articles, for example, diapers, incontinence products, and feminine hygiene products. It is desirable for the absorbent articles to have a high absorbent capacity for liquid, as well as to have good strength characteristics for durability. In addition to absorbent capacity, the ability to rapidly absorb a liquid is a desirable characteristic of an absorbent article. For example, diapers and other hygienic products that do not contain a dedicated liquid acquisition component suffer from measurable urine containment problems as well as rewet, that is, the feeling of dampness to touch after use.

One solution to the problem of providing absorbent articles that possess the advantageous properties of high absorbent capacity, rapid liquid acquisition, and superior rewet performance has been the production of absorbent articles that combine an acquisition layer with one or more other layers. For example, the combination of one layer having rapid liquid acquisition characteristics with another layer having high absorbent capacity results in a product that offers the advantages of both layers.

A recognized problem with conventional acquisition layers is their tendency to collapse upon wetting. Such a wet collapse impairs the permeability of the structure and can result in liquid leakage from the absorbent article.

Another recognized problem with cellulose-based acquisition layers that are air laid on diaper lines is their relatively poor dry and wet integrity. Upon movement and/or wetting, the acquisition layers can crack, bunch, and disintegrate, all of which adversely affect fluid transfer between the layers and significantly impact the layers' fluid-handling capability. Furthermore, consumers react negatively to bunched diapers.

It has also been recognized that forming fibrous webs that contain high levels of crosslinked cellulosic fibers and/or in combination with synthetic fibers is difficult because of the flocculent nature of the fibers. In addition, due to the low density of the fibers, large quantities of such webs having appreciable roll life for diaper line production are difficult to provide.

Accordingly, there exists a need for an acquisition layer that can be incorporated into an absorbent article that has enhanced dry and wet integrity, increased resistance to wet collapse, and provides increased permeability and porosity to effect the rapid acquisition and distribution of acquired liquid and improved rewet performance. A need also exists for delivering such a material in a form that reduces the material handling problems associated with bulky webs. The present invention seeks to fulfill these needs and provides further related advantages.

### SUMMARY OF THE INVENTION

The present invention is a unitary absorbent layer that includes a fibrous material and a binder. In a preferred embodiment, the absorbent layer includes a thermally bonded mixture of crosslinked cellulose fibers and multicomponent binding fibers. In combination with one or more other layers in an absorbent article, the unitary absorbent layer can rapidly acquire, distribute, temporarily store, and then release the acquired liquid to other liquid retention layers. The unitary absorbent layer can be formed by foam forming processes.

### BRIEF DESCRIPTION OF THE DRAWINGS

The foregoing aspects and many of the attendant advantages of this invention will become more readily appreciated by reference to the following detailed description, when taken in conjunction with the accompanying drawings, wherein:

FIG. 1 is a schematic view of one absorbent article incorporating a unitary absorbent layer produced in accordance with the present invention;

FIG. 2 is a schematic view of another absorbent article incorporating a unitary absorbent layer produced in accordance with the present invention;

FIG. 3 is a schematic view of still another absorbent article incorporating a unitary absorbent layer produced in accordance with the present invention;

FIG. 4 is a schematic view of yet another absorbent article incorporating a unitary absorbent layer produced in accordance with the present invention;

FIG. 5 is a schematic view of another absorbent article incorporating a unitary absorbent layer produced in accordance with the present invention;

FIG. 6 is a graph comparing the acquisition time and rewet performance of a diaper incorporating a representative unitary absorbent layer formed in accordance with the present invention; and

FIGS. 7A and 7B are photographs of a representative unitary absorbent layer formed in accordance with the present invention and a wet-laid absorbent layer, respectively.

### DETAILED DESCRIPTION OF THE PREFERRED EMBODIMENT

In one aspect, the present invention provides a unitary absorbent layer that includes a fibrous material and a binder. Generally, the fibrous material includes one or more hydrophilic fibers and, optionally, additional fibers such as hydrophobic fibers including synthetic fibers. The unitary absorbent layer of this invention has increased wet and dry integrity and improved pore size uniformity compared to conventional acquisition layers. The unitary absorbent layer of the present invention can be incorporated into a variety of absorbent products and articles to increase the liquid acquisition rate, improve the rewet performance, and enhance the wet and dry integrity of the absorbent article. Thus, the unitary absorbent layer is an absorbent layer that is useful as an acquisition layer in absorbent products.

In another aspect of the present invention, a foam forming method for producing a unitary absorbent layer is provided.

In addition to serving as an acquisition layer that can rapidly acquire fluid and reduce rewet, because of increased permeability and pore size uniformity, the unitary absorbent layer of the invention can also serve as a distribution layer that transports liquid from the site of insult throughout the composite, and then ultimately to a highly absorbent core or permanent retention layer. Furthermore, because of the substantial absorbent capacity of the composite's fibrous material, the unitary absorbent layer can also serve as a storage layer. Thus, when configured in combination with other layers in an absorbent construct, the unitary absorbent layer serves as a temporary storage layer that can rapidly release liquid to other core or retention layers. As used herein, the term “temporary storage” refers to the ability of a material to temporarily provide holding capacity for a liquid until an external force drains the fluid from the material. The external force can be, for example, greater capillary pressure or otherwise exerted by an adjacent storage layer.

Generally, the unitary absorbent layer of the present invention includes a fibrous material in combination with a binder. As used herein, the term “fibrous material” refers to any material that includes one or more hydrophilic fibers and, optionally, additional fibers such as hydrophobic fibers including synthetic fibers. Synthetic and/or hydrophobic fibers can also be included in the absorbent layer, provided that the overall composite remains relatively hydrophilic and maintains the advantageous properties of wet integrity and permeability characteristic of the unitary absorbent layer of the present invention. In a preferred embodiment, the hydrophilic fibers include cellulosic fibers, and more preferably crosslinked cellulosic fibers. Suitable and preferred cellulosic fibers are described below. Cellulosic fibers can be present in the layer in an amount from about 5% to about 95%, preferably from about 70% to about 90%, by weight of the total layer.

In addition to the cellulosic fibers noted above, synthetic fibers can also be included in the unitary absorbent layer of the present invention. Suitable synthetic fibers include, for example, polyethylene terephthalate (PET), polyethylene, polypropylene, nylon, and rayon fibers.

For the unitary absorbent layers of this invention that include synthetic fibers, the performance of the composite has been found to be dependent upon a number of factors including the length, denier (g/m), and physical nature of the synthetic fibers. Suitable synthetic fibers useful in forming the acquisition composite can have a length up to about 2 inches, and preferably have a length between about 0.25 and about 1.5 inches. One advantage of the foam method for forming the unitary absorbent layer of the invention is that, unlike air-laid and wet-laid methods, relatively long fibers can be readily accommodated by the process. Suitable fibers include fibers having denier up to about 40 denier, and preferably between about 5 and about 20 denier. While straight fibers can be advantageously used in the formation of the acquisition composite, in a preferred embodiment, the fibers are crimped.

Cellulosic fibers are a basic component of the unitary absorbent layer of the present invention. Although available from other sources, cellulosic fibers are derived primarily from wood pulp. Suitable wood pulp fibers for use with the invention can be obtained from well-known chemical processes such as the Kraft and sulfite processes, with or without subsequent bleaching. The pulp fibers may also be processed by thermomechanical, chemithermomechanical methods, or combinations thereof. The preferred pulp fiber is produced by chemical methods. Ground wood fibers, recycled or secondary wood pulp fibers, and bleached and unbleached wood pulp fibers can be used. The preferred starting material is prepared from long fiber coniferous wood species, such as southern pine, Douglas fir, spruce, and hemlock. Details of the production of wood pulp fibers are well-known to those skilled in the art. These fibers are commercially available from a number of companies, including Weyerhaeuser Company, the assignee of the present invention. For example, suitable cellulose fibers produced from southern pine that are usable with the present invention are available from Weyerhaeuser Company under the designations CF416, NF405, PL416, FR516, and NB416.

The wood pulp fibers useful in the present invention can also be pretreated prior to use with the present invention. This pretreatment may include physical treatment, such as subjecting the fibers to steam, or chemical treatment, for example, crosslinking the cellulose fibers using any of a variety of conventional crosslinking agents such as dimethyldihydroxyethyleneurea. Crosslinking the fibers, for example, increases their resiliency, and thereby can improve their absorbency. The fibers may also be twisted or crimped, as desired. Suitable crosslinked cellulose fibers produced from southern pine are available from Weyerhaeuser Company under the designation NHB416. Crosslinked cellulose fibers and methods for their preparation are disclosed in U.S. Pat. No. 5,225,047, issued Jul. 6, 1993, entitled “Crosslinked Cellulose Products and Method For Their Preparation,” expressly incorporated herein by reference.

Although not to be construed as a limitation, examples of pretreating fibers include the application of fire retardants to the fibers, and surfactants or other liquids, such as water or solvents, which modify the surface chemistry of the fibers. Other pretreatments include incorporation of antimicrobials, pigments, and densification or softening agents. Fibers pretreated with other chemicals, such as thermoplastic and thermosetting resins also may be used. Combinations of pretreatments also may be employed. Similar treatments can also be applied after the composite formation in post-treatment processes.

Cellulosic fibers treated with particle binders and/or densification/softness aids known in the art can also be employed in accordance with the present invention. The particle binders serve to attach other materials, such as cellulosic fiber superabsorbent polymers as well as others, to the cellulosic fibers. Cellulosic fibers treated with suitable particle binders and/or densification/softness aids and the process for combining them with cellulose fibers are disclosed in the following U.S. patents and patent applications: (1) U.S. Pat. No. 5,543,215, entitled “Polymeric Binders for Binding Particles to Fibers”; (2) U.S. Pat. No. 5,538,783, entitled “Non-Polymeric Organic Binders for Binding Particles to Fibers”; (3) U.S. Pat. No. 5,300,192, entitled “Wet Laid Fiber Sheet Manufacturing With Reactivatable Binders for Binding Particles to Binders;” (4) U.S. Pat. No. 5,352,480, entitled “Method for Binding Particles to Fibers Using Reactivatable Binders”; (5) U.S. Pat. No. 5,308,896, entitled “Particle Binders for High-Bulk Fibers”; (6) U.S. patent application Ser. No. 07/931,279, filed Aug. 17, 1992, entitled “Particle Binders that Enhance Fiber Densification”; (7) U.S. patent application Ser No. 08/107,469, filed Aug. 17, 1993, entitled “Particle Binders”; (8) U.S. patent application Ser. No. 08/108,219, filed Aug. 17, 1993, entitled “Particle Binding to Fibers”; (9) U.S. patent application Ser. No. 08/107,467, filed Aug. 17, 1993, entitled “Binders for Binding Water Soluble Particles to Fibers”; (10) U.S. Pat. No. 5,547,745, entitled “Particle Binders”; (11) U.S. patent application Ser. No. 08/108,218. filed Aug. 17, 1993, entitled “Particle Binding to Fibers”; and (12) U.S. Pat. No. 5,308,896, entitled “Particle Binders for High-Bulk Fibers,” all expressly incorporated herein by reference. One example of a suitable densification/softness aid is a mixture of 70% sorbitol and 30% glycerin. The composite is treated with sorbitol and glycerin by spraying the composite with the mixture and passing the composite through a roll coater, or other means familiar to those skilled in the art of adding a liquid to a composite.

Materials that enhance absorbent capacity, such as superabsorbent polymers, can also be combined with the unitary absorbent layer of the present invention. A superabsorbent polymer as used herein is a polymeric material that is capable of absorbing large quantities of fluid by swelling and forming a hydrated gel (hydrogel). The superabsorbent polymers also can retain significant amounts of bodily fluids under moderate pressures. Superabsorbent polymers generally fall into three classes, namely, starch graft copolymers, crosslinked carboxymethylcellulose derivatives and modified hydrophilic polyacrylates. Examples of such absorbent polymers are hydrolyzed starch-acrylonitrile graft copolymer, a neutralized starch-acrylic acid graft copolymer, a saponified acrylic acid ester-vinyl acetate copolymer, a hydrolyzed acrylonitrile copolymer or acrylamide copolymer, a modified crosslinked polyvinyl alcohol, a neutralized self-crosslinking polyacrylic acid, a crosslinked polyacrylate salt, carboxylated cellulose, and a neutralized crosslinked isobutylene-maleic anhydride copolymer. The superabsorbent polymeric materials can be combined with the layer's fibers in amounts up to about 5%, and preferably about 2%, by weight based on the total weight of the layer.

Superabsorbent polymers are available commercially, for example, starch graft polyacrylate hydrogel fines from Hoechst Celanese of Portsmouth, Va. These superabsorbent polymers come in a variety of sizes, morphologies, and absorbent properties. These are available from Hoechst Celanese under trade designations such as IM 1000 and IM 3500. Other superabsorbent particles are marketed under the trademarks SANWET (supplied by Sanyo Kasei Kogyo Kabushiki Kaisha), SUMIKA GEL (supplied by Sumitomo Kagaku Kabushiki Kaisha), which is suspension polymerized and spherical, as opposed to solution polymerized ground particles, FAVOR (supplied by Stockhausen of Greensboro, N.C.), and NORSOCRYL (supplied by Atochem). Other superabsorbent polymers are described in U.S. Pat. No. 4,160,059; U.S. Pat. No. 4,676,784; U.S. Pat. No. 4,673,402; U.S. Pat. No. 5,002,814; U.S. Pat. No. 5,057,166; U.S. Pat. No. 4,102,340; and U.S. Pat. No. 4,818,598, expressly incorporated herein by reference. Products such as diapers that incorporate superabsorbent polymers are shown in U.S. Pat. No. 3,669,103 and U.S. Pat. No. 3,670,731.

The unitary absorbent layer of the present invention is formed by combining a fibrous material (i.e., one or more hydrophilic fibers optionally in combination with one or more hydrophobic and/or synthetic fibers) with a binder. As used herein, the term “binder” refers to a system that is effective in intertwining and/or bonding the fibers to each other and/or the fibers of the binder. Suitable binders include bonding agents such as thermoplastic and thermosetting bonding agents, soluble bonding mediums used in combination with solvents, and wet strength agents. Alternatively, the absorbent layer's hydrophilic fibers can be intertwined and/or bonded through a mechanical process including, for example, hydroentanglement, embossing, tenderizing, and needling processes.

Suitable binders include bonding agents, such as cellulosic and synthetic fibrous materials, and soluble bonding mediums as described below. In one preferred embodiment, the binder is a synthetic fibrous material, such as Celbond® (Hoechst Celanese) and D-271P® (DuPont). In another preferred embodiment, the binder includes a soluble bonding medium, more preferably cellulose acetate used in combination with the solvent triacetin. Generally, the binder is included in the composite in an amount up to about 30%, and preferably about 20%, by weight of the total composite.

Bonding agents useful in the binder in accordance with the present invention are those materials that (a) are capable of being combined with and dispersed throughout a web of cellulosic fibers, (b) when activated, are capable of coating or otherwise adhering to the fibers or forming a binding matrix, and (c) when deactivated, are capable of binding at least some of the fibers together. The use of bonding agents with cellulose fiber webs is disclosed in U.S. patent application Ser. No. 08/337,642, filed Nov. 10, 1994, entitled “Densified Cellulose Fiber Pads and Methods of Making the Same,” expressly incorporated herein by reference.

Suitable bonding agents include thermoplastic materials that are activated by melting at temperatures above room temperature. When these materials are melted, they will coat at least portions of the cellulose fibers with which they are combined. When the thermoplastic bonding agents are deactivated by cooling to a temperature below their melt point, and preferably no lower than room temperature, the bonding agent will, upon solidifying from the melted state, cause the cellulose fibers to be bound in a matrix.

Thermoplastic materials are the preferred binders, and can be combined with the fibers in the form of particles, emulsions, or as fibers. Suitable fibers can include those made from thermoplastic polymers, cellulosic or other fibers coated with thermoplastic polymers, and multicomponent fibers in which at least one of the components of the fiber comprises a thermoplastic polymer. Single and multicomponent fibers are manufactured from polyester, polyethylene, polypropylene and other conventional thermoplastic fiber materials. The same thermoplastics can be used in particulate or emulsion form. Many single-component fibers are commercially available. Suitable multicomponent fibers include Celbond® fibers, a bicomponent fiber, available from Hoechst Celanese Company. Suitable coated fibers can include cellulose fibers coated with latex or other thermoplastics, as disclosed in U.S. Pat. No. 5,230,959, issued Jul. 27, 1993, to Young et al., and U.S. Pat. No. 5,064,689, issued Nov. 12, 1991, to Young et al. The thermoplastic fibers are preferably combined with the cellulose fibers before or during the laying process. When used in particulate or emulsion form, the thermoplastics can be combined with the cellulose fibers before, during, or after the laying process.

Other suitable thermoplastic bonding agents include ethylene vinyl alcohol, polyvinyl acetate, acrylics, polyvinyl acetate acrylate, polyvinyl dichloride, ethylene vinyl acetate, ethylene vinyl chloride, polyvinyl chloride, styrene, styrene acrylate, styrene butadiene, styrene acrylonitrile, butadiene acrylonitrile, acrylonitrile butadiene styrene, ethylene acrylic acid, urethanes, polycarbonate, polyphenylene oxide, and polyimides.

Thermosetting materials also serve as excellent bonding agents for the present invention. Typical thermosetting materials are activated by heating to elevated temperatures at which crosslinking occurs. Alternatively, a resin can be activated by combining it with a suitable crosslinking catalyst before or after it has been applied to the cellulosic fiber. Thermosetting resins can be deactivated by allowing the crosslinking process to run to completion or by cooling to room temperature, at which point crosslinking ceases. When crosslinked, it is believed that the thermosetting materials form a matrix to bond the cellulose fibers. It is contemplated that other types of bonding agents can also be employed, for example, those that are activated by contact with steam, moisture, microwave energy, and other conventional means of activation.

Thermosetting bonding agents suitable for the present invention include phenolic resins, polyvinyl acetates, urea formaldehyde, melamine formaldehyde, and acrylics. Other thermosetting bonding agents include epoxy, phenolic, bismaleimide, polyimide, melamine formaldehyde, polyester, urethanes, and urea.

These bonding agents are normally combined with the fibers in the form of an aqueous emulsion. They can be combined with the fibers during the laying process. Alternatively, they can be sprayed onto a loose web after it has been formed.

As noted above, the binder utilized in accordance with the present invention can also be a soluble bonding medium that can be incorporated with the pulped cellulosic fibers, either in fiber form, or as particles or granules. If desired, the bonding medium can also be coated onto solvent-insoluble fibers, such as cellulosic fibers, which can then be distributed throughout the matrix of pulped cellulosic fibers. It is presently preferred that the bonding medium comprise a fiber and be mixed with the pulped cellulosic fibers during, for example, the formation of a fluff web by conventional air-laid processes. The use of soluble bonding mediums with cellulose fiber webs is disclosed in U.S. patent application Ser. No. 08/669,406, filed Jul. 3, 1996, entitled “Fibrous Web Having Improved Strength and Method of Making the Same,” expressly incorporated herein by reference.

The solvents employed in accordance with the present invention must of course be capable of partially solubilizing the bonding medium as described above. The solvents must be able to partially dissipate or migrate from the surface of the bonding medium to allow the bonding medium to resolidify after partial solubilization. Nonvolatile solvents may be dissipated in most part by absorption into the bonding medium. It is preferred that the solvent be of limited volatility, so that little or no solvent will be lost to the atmosphere. By limited volatility it is meant that the solvent has a vapor pressure of 29 kPa or less at 25° C. Using a solvent of limited volatility may mitigate precautions usually necessary to control volatiles, and reduces the amount of solvent required to partially solubilize the bonding medium. In addition, use of solvents of limited volatility may eliminate the attendant processing problems encountered with volatile solvents, many of which are flammable and must be handled with care. The use of solvents of limited volatility may also reduce environmental problems. Furthermore, it is desirable for solvents to be nontoxic and capable of being dissipated from the surface of the bonding medium without adversely affecting the overall strength of the bonding medium.

Preferred bonding mediums and solvents of limited volatility are listed in the table set forth below.

| Bonding Medium | Solvent |
|---|---|
| cellulose acetate | tracetin |
| | propane diol diacetate |
| | propane diol |
| | dipropionate |
| | propane diol dibutyrate |
| | triethyl citrate |
| | dibutyl phthalate |
| cellulose nitrate | triacetin |
| cellulose butyrate | triacetin |
| vinyl chloride/vinyl acetate copolymer | triacetin |
| cellulose fibers coated with polyvinyl acetate | triacetin |

Of the several bonding mediums listed, cellulose acetate is the most preferred. During manufacture of cellulose acetate fibers, a finish is usually applied to the fibers. Many times this finish is in the form of an oil. The presence of the finish sometimes detracts from the performance of a bonding medium. The presence of a finish may adversely affect the development as well as the strength of the bonds. It has been found that when the bonding fibers are as straight as possible, as opposed to curled or kinked, they provide more contact points with the cellulosic fibers, and thus the final web will develop better strength. Similarly, when the bonding fibers are as long as is reasonably possible, the strength of the final web is increased. In addition to the foregoing, cellulose ethers and other cellulose esters may also be used as bonding medium. Acetylated pulp fibers may also be used as bonding medium and may be substituted with any number of acetyl groups. A preferred degree of substitution (D.S.) would be 2 to 3, and a most preferred D.S. would be 2.4.

The solvents used in combination with the bonding medium can be added in varying amounts. Strength is adversely affected if too little or too much solvent is added. At a cellulose acetate/pulp weight ratio of 10:90, it has been found that the solvents, and particularly triacetin, provide good strength when added in amounts ranging from 6% to 17%, and most preferably in the range of 9% to 14%, based on the weight of pulp fiber present.

The preferred forms of the solvents propane diol diacetate, dipropionate, and dibutyrate are the 1, 2 and 1, 3 forms. Other suitable solvents that work in accordance with present invention are butyl phthalyl butyl glycolate, N-cyclohexyl-p-toluenesulfonamide, diamyl phthalate, dibutyl phthalate, dibutyl succinate, dibutyl tartrate, diethylene glycol dipropionate, di-(2-ethoxyethyl) adipate, di-(2-ethoxyethyl) phthalate, diethyl adipate, diethyl phthalate, diethyl succinate, diethyl tartrate, di-(2-methoxyethyl) adipate, di-(2-methoxyethyl) phthalate, dimethyl phthalate, dipropyl phthalate, ethyl o-benzoylbenzoate, ethyl phthalyl ethyl glycolate, ethylene glycol diacetate, ethylene glycol dibutyrate, ethylene glycol dipropionate, methyl o-benzoylbenzoate, methyl phthalyl ethyl glycolate, N-o and p-tolylethylsulfonamide, o-tolyl p-toluenesulfonate, tributyl citrate, tributyl phosphate, tributyrin, triethylene glycol diacetate, triethylene glycol dibutyrate, triethylene glycol dipropionate, and tripropionin.

The binder useful in the composite of this invention can also include polymeric agents that can coat or impregnate cellulosic fibers. Suitable agents include cationic modified starch having nitrogen-containing groups (e.g., amino groups) such as those available from National Starch and Chemical Corp., Bridgewater, N.J.; latex; wet strength agents such as polyamide-epichlorohydrin resin (e.g., Kymene™ 557H, Hercules, Inc., Wilmington, Del.), polyacrylamide resin (described, for example, in U.S. Pat. No. 3,556,932 issued Jan. 19, 1971 to Coscia et al.; and commercially available polyacrylamide marketed by American Cyanamid Co., Stanford, Conn., under the designation Parez™, for example, Parez™ 631 NC); urea formaldehyde and melamine formaldehyde resins, and polyethylenimine resins. A general discussion on wet strength resins utilized in the paper field, and generally applicable in the present invention, can be found in TAPPI monograph series No. 29, “Wet Strength in Paper and Paperboard”, Technical Association of the Pulp and Paper Industry (New York, 1965). Other binders include adhesive systems and scrim. For embodiments of the unitary absorbent layer that include a wet strength agent as a binder, the wet strength agent is present in the layer in an amount from about 0.1% to about 2.0%, preferably from about 0.5% to about 1.0%, by weight of the total layer.

Preferably, the binder is integrally incorporated into or onto the hydrophilic fibrous web that is formed in the production of the unitary absorbent layer. The binder can be added to pulp prior to web formation, by applying the binder to the foam-formed web after web deposition, after drying, or a combination thereof

Additives can also be incorporated into a unitary absorbent layer of the present invention during composite formation. The advantage of incorporating the additives during composite formation is that they will also be attached to the acquisition matrix by certain of the solvents and bound in the matrix by the bonding medium. This provides a significant advantage in that the additives can be dispersed and retained throughout the matrix where desired. For example, the additives may be evenly dispersed and retained throughout the matrix. Additives that can be incorporated into the matrix include absorbent capacity enhancing materials such as superabsorbent polymers, adsorbents such as clays, zeolites, and activated carbon, brighteners such as titanium oxide, and odor absorbents such as sodium bicarbonate. Solvents can also reduce the dusting caused by the additives or the pulp itself because more of the fines are attached and bound to the matrix by the bonding medium.

In another aspect, the present invention provides methods for producing a unitary absorbent layer by foam-forming processes. A unitary absorbent layer can be produced in accordance with the present invention by foam processes known in the art. See, for example, U.S. Pat. Nos. 3,716,449; 3,839,142; 3,871,952; 3,937,273; 3,938,782; 3,947,315; 4,166,090; 4,257,754; and 5,215,627, assigned to Wiggins Teape and related to the formation of fibrous materials from foamed aqueous fiber suspensions, and “The Use of an Aqueous Foam as a Fiber-Suspending Medium in Quality Papermaking,” Foams, Proceedings of a Symposium organized by the Society of Chemical Industry, Colloid and Surface Chemistry Group, R. J. Akers, Ed., Academic Press, 1976, which describes the Radfoam process, all expressly incorporated herein by reference.

Generally, in one embodiment, the methods for forming the unitary absorbent layer of this invention include combining a fibrous material with a binder, followed by depositing the resulting fibrous mixture onto a foraminous support. Alternatively, a web of fibrous material may be treated with a suitable binder. In any event, the deposited web containing the fibrous material and binder is then subjected to conditions sufficient to effect bonding (i.e., thermal bonding) between the fibrous material and the binder to provide the unitary absorbent layer of the invention.

For foam-formed methods, the fibrous mixture is an aqueous foam slurry that includes a surfactant. Suitable surfactants include ionic, nonionic, and amphoteric surfactants known in the art. In the method, the deposited slurry is a water-containing composite and these methods include the step of removing at least some portion of water from the composite deposited on the foraminous support. The deposited composite is then subjected to conditions, for example, heating, to effect drying and thermal bonding of the fibers.

The unitary absorbent layer of the present invention is prepared by a foam forming process. For fabrication, the unitary absorbent layer is formed by a foam process, preferably a process by Ahlstrom Company (Helsinki, Finland). This process encompasses desirable manufacturing efficiencies while producing a product with excellent performance. The formation of a representative unitary absorbent layer of the present invention by representative foam processes is described in Examples 1 and 2. The performance characteristics of representative unitary absorbent layers produced by the methods noted above are described in Examples 4 and 5.

Foam forming methods provide fibrous webs that possess both relatively low density and relatively high tensile strength. For webs composed of substantially the same components, foam-formed webs generally have densities greater than air-laid webs and lower than wet-laid webs. Similarly, the tensile strength of foam formed webs is substantially greater than for air-laid webs and approach the strength of wet-laid webs. For fibrous webs that are thermally consolidated, for example, webs that include bicomponent binding fibers that effect interfiber bonding upon heat treatment, tensile strength is less dependent on the method for forming. However, for such webs, their density can vary depending on the method of forming.

For example, the wet and dry density of a representative unitary absorbent layer of the present invention is compared to wet-laid absorbent layer in the table below. Both layers are a thermally bonded blend of crosslinked cellulosic fibers (80% by weight) and bicomponent binding fibers (20% by weight).

| Formation Method | Dry (g/cm³) | Wet (g/cm³) |
|---|---|---|
| Wet Laid | 0.042 | 0.084 |
| FoamFormed | 0.033 | 0.081 |

The dry tensile strengths of the representative unitary absorbent layer of the present invention and the wet-laid absorbent layer noted above and having basis weights of 70 g/m² are compared in the table below.

| Formation Method | MD dry (g/in) |
|---|---|
| Wet Laid | 431 |
| Foam Formed | 497 |

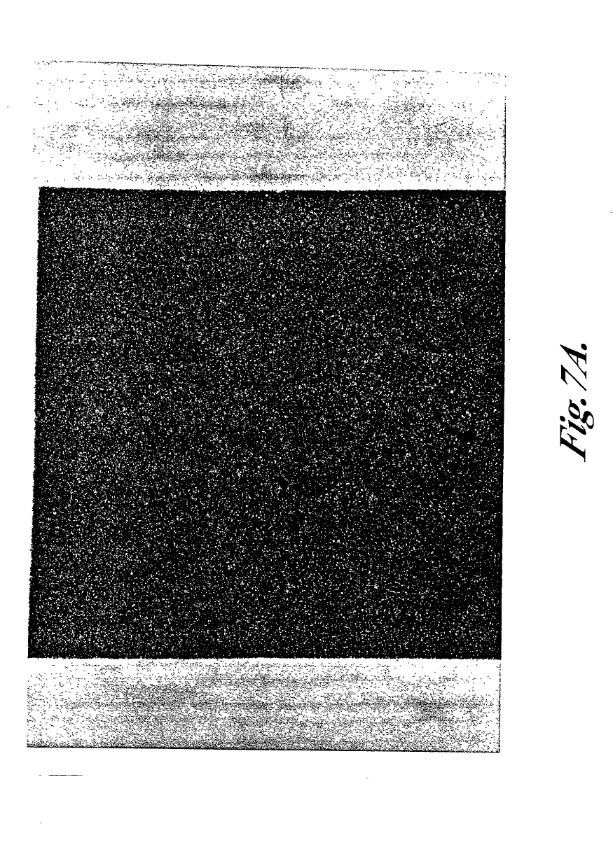

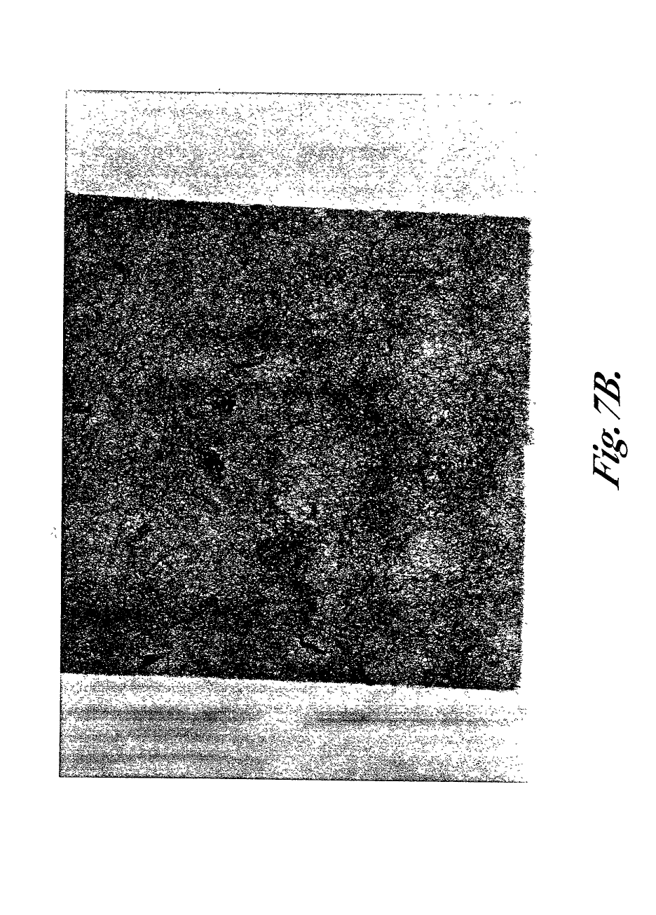

Web density reflects web pore size. The pore size of an air-laid web is generally greater than the pore size of a wet-laid web. The pore size of webs formed by foam processes is generally greater than for wet-laid webs and less than for air-laid webs. In addition to providing greater control over the characteristics of webs formed by the process, foam processes generally provide fibrous webs having substantially uniform pore size compared to air- and wet-laid webs. The difference in the uniformity of the representative unitary absorbent layer and the wet-laid absorbent layer noted above is illustrated in FIGS. 7A and 7B, respectively. As shown in FIG. 7A, the foam formed layer is significantly more homogeneous than the comparable wet-laid layer shown in FIG. 7B. The improved formation of the unitary absorbent layer relative to the wet-laid layer is apparent from the figure. The uniformity of the foam formed layer is unexpected considering that the fibers of the layer are crosslinked cellulosic fibers which, because of their morphology, generally provide relatively high bulk, resilient, and nonhomogeneous structures.

The unitary absorbent layer of the present invention generally has a basis weight from about 10 to about 1500 g/m², and preferably from about 20 to about 500 g/m². In a more preferred embodiment, the absorbent layer has a basis weight in the range from about 40 to about 400 g/m².

Generally, the unitary absorbent layer has a density from about 0.02 to about 0.2 g/cm³, and preferably from about 0.04 to about 0.10 g/m³. In one embodiment, the unitary absorbent layer is a densified composite. Generally, densified products have improved liquid distribution properties relative to undensified products. Densification methods useful in producing the densified composites of the present invention are well known to those in the art. See, for example, U.S. patent application Ser. No. 08/337,642, filed Nov. 10, 1994, entitled “Densified Cellulose Fiber Pads and Methods of Making the Same,” expressly incorporated herein by reference. Densified unitary absorbent layers of this invention generally have a density from about 0.1 to about 0.6 g/cm³, and preferably from about 0.2 to about 0.4 g/m³.

Preferably, the unitary absorbent layer of the invention is an undensified composite. Accordingly, production methods used in connection with the absorbent layer preferably do not include subjecting the absorbent layer, or absorbent articles that incorporate the absorbent layer, to densification conditions. For example, in the production of diapers that incorporate the absorbent layer of the present invention, the absorbent layer is preferably incorporated into the diaper after the diaper has been subjected to the application of pressure such as, for example, being passed through a calender roll.

The unitary absorbent layer can be produced in a number of forms including sheets and rolls, and having a variety of thicknesses.

The unitary absorbent layer of the invention is generally characterized as having increased wet integrity (i.e., increase resistance to wet collapse) compared to conventional acquisition layers. The increased wet pad integrity of the absorbent layer of this invention prevents wet collapse and tearing of the composite during liquid insult and thereby avoids leakage during insult from absorbent articles that incorporate the acquisition layer. For example, a representative wet-laid acquisition layer formed in accordance with the present invention having a basis weight of 300 g/m² had a wet tensile strength of about 400 g/inch. Similarly, other representative wet-laid acquisition layers having basis weights of 50 and 40 g/m² had tensile strengths of 120 and 90 g/inch, respectively. In comparison, the tensile strength of an air-laid acquisition layer made of 100% crosslinked fibers and having a basis weight of 300 g/m² was below the detection limit of the tensile strength determining method. The tensile strength determination is described in Example 5.

The unitary absorbent layer of the invention also has increased pore size uniformity compared to conventional acquisition layers. The composite's uniform pore size is maintained during liquid insult and thereby effectively facilitates transport and distribution of the acquired liquid from the point of initial insult to other portions of the composite and, ultimately, to the absorbent article's core or permanent storage layer where the liquid is finally absorbed.

The unitary absorbent layers of the present invention are generally softer to the touch than comparably composed wet-laid layers. The softness of the representative unitary absorbent layer of the present invention and the wet-laid absorbent layer noted above are compared in the table below. Softness was evaluated by a panel of 25 persons who ranked various materials on a softness index scale from 1 (rough surface feel) to 10 (soft surface feel).

| Formation Method | Soft Index |
|---|---|
| Wet Laid | 3.7 |
| Foam Formed | 5.4 |

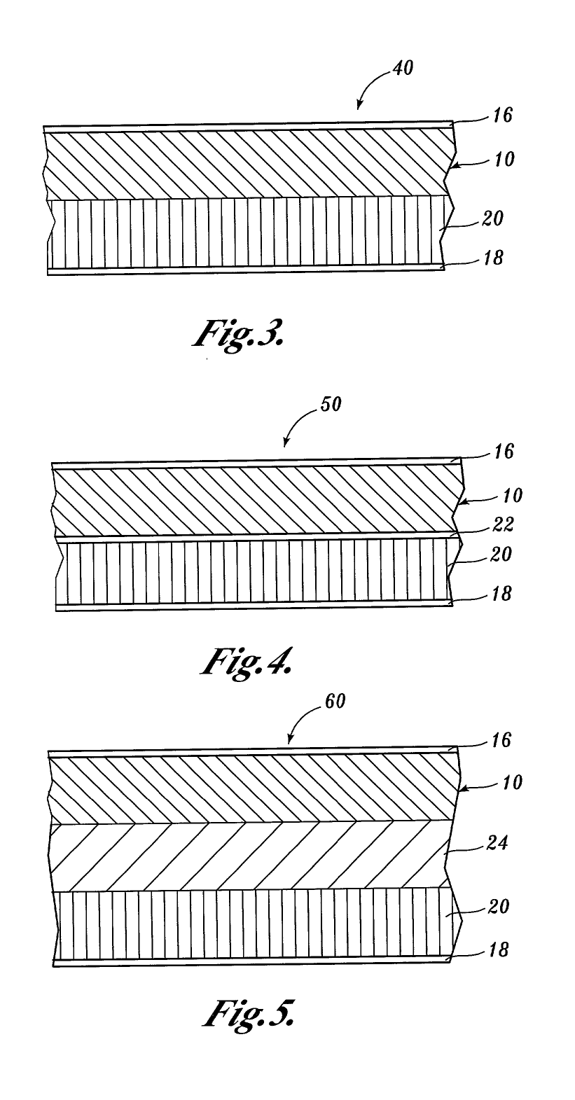

Depending upon the nature of the absorbent construct, an absorbent article incorporating the unitary absorbent layer may include one or more additional layers, such as a core layer (i.e., permanent storage layer) (see, for example, FIGS. 3-5). In such a construct, in addition to rapidly absorbing the acquired liquid, the acquisition composite has absorbent capacity sufficient to temporarily hold the acquired liquid and therefore provide time sufficient for the core layer to permanently absorb liquid from the acquisition composite.

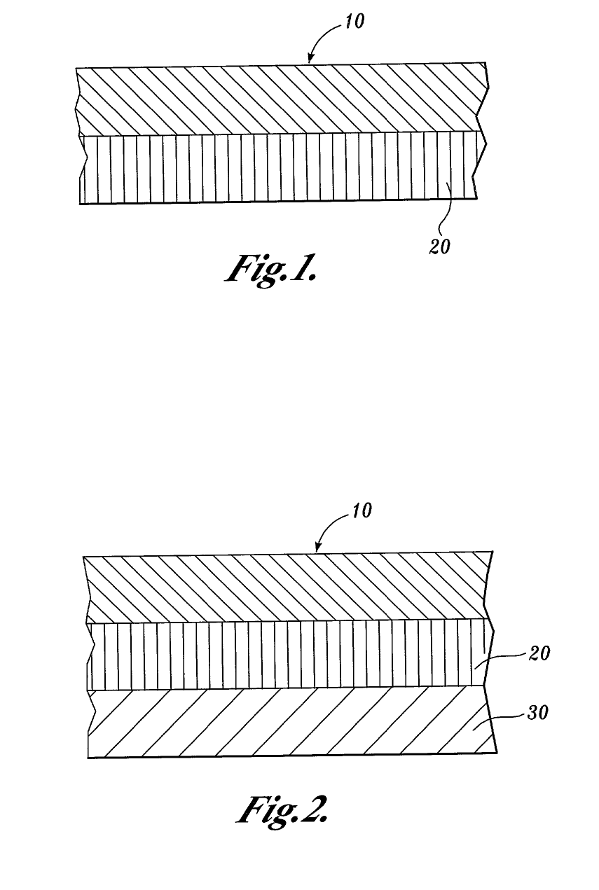

As noted above, the unitary absorbent layer can be incorporated in an absorbent article as an absorbent acquisition/distribution layer. The absorbent layer can be used alone, or as illustrated in FIG. 1, can be used in combination with one or more secondary layers. In FIG. 1, unitary absorbent layer 10 is employed as an upper acquisition/distribution layer in combination with a storage layer 20 composed of, for example, a fibrous web. Storage layer 20, if desired, can also comprise a densified layer of bonded cellulose fibers. As illustrated in FIG. 2, a third layer 30 (e.g., a core or retention layer) can also be employed, if desired, with a storage layer 20 and unitary absorbent layer 10. If desired, the retention layer 30 can also be composed of a fibrous web such as, for example, densified bonded cellulose fibers.

A variety of suitable constructs can be produced from the unitary absorbent layer. The most common include absorptive consumer products such as diapers, feminine hygiene products such as feminine napkins, and adult incontinence products. For example, referring to FIG. 3, an absorbent article 40 comprises unitary absorbent layer 10 and an underlying storage layer 20. A liquid pervious facing sheet 16 overlies unitary absorbent layer 10, and a liquid impervious backing sheet 18 underlies the storage layer 20. The unitary absorbent layer will provide advantageous liquid acquisition performance for use in, for example, diapers. The capillary structure (i.e., pore size, pore size uniformity, and permeability) of the acquisition composite will aid in fluid transport in multiple wettings. Generally, the storage layer 20 includes a fibrous web, for example, a strengthened web of cellulose fibers, and may also incorporate additives, such as superabsorbent polymers to significantly increase the absorbent capacity of the storage layer 20.

The article of FIG. 3 can be assembled such that unitary absorbent layer 10 is brought into contact with the storage layer 20 while the binder in the latter is still active. Such a procedure will allow the storage layer to bond to at least the lower surface of layer 10, and thus eliminate the need to use hot-melt glues to bond adjacent layers.

A stronger bond between layer 10 and the storage layer 20 can be achieved by contacting the layer with the storage layer while the layer's binder is still active. Similarly, laying the storage layer 20 on the backing sheet 18 while the binder of the storage layer is still active results in the bonding of layer 20 to the backing sheet 18. In a similar manner, layer 10 may be bonded to the facing sheet 16 by laying the facing sheet on layer 10 while the binder therein is still active. Interbonding between layers can generally enhance and further facilitate fluid transport across the layer interface.

The construct in FIG. 3 is shown for purposes of exemplifying a typical absorbent article, such as a diaper or feminine napkin. One of ordinary skill will be able to make a variety of different absorbent constructs using the concepts taught herein. For example, a typical construction for an adult incontinence absorbent structure is shown in FIG. 4. The article 50 comprises a facing sheet 16, unitary absorbent layer 10, a storage layer 20, and a backing sheet 18. The facing sheet 16 is pervious to liquid while the backing sheet 18 is impervious to liquid. In this construct, a liquid pervious tissue 22 composed of a polar, fibrous material is positioned between acquisition composite 10 and storage layer 20.

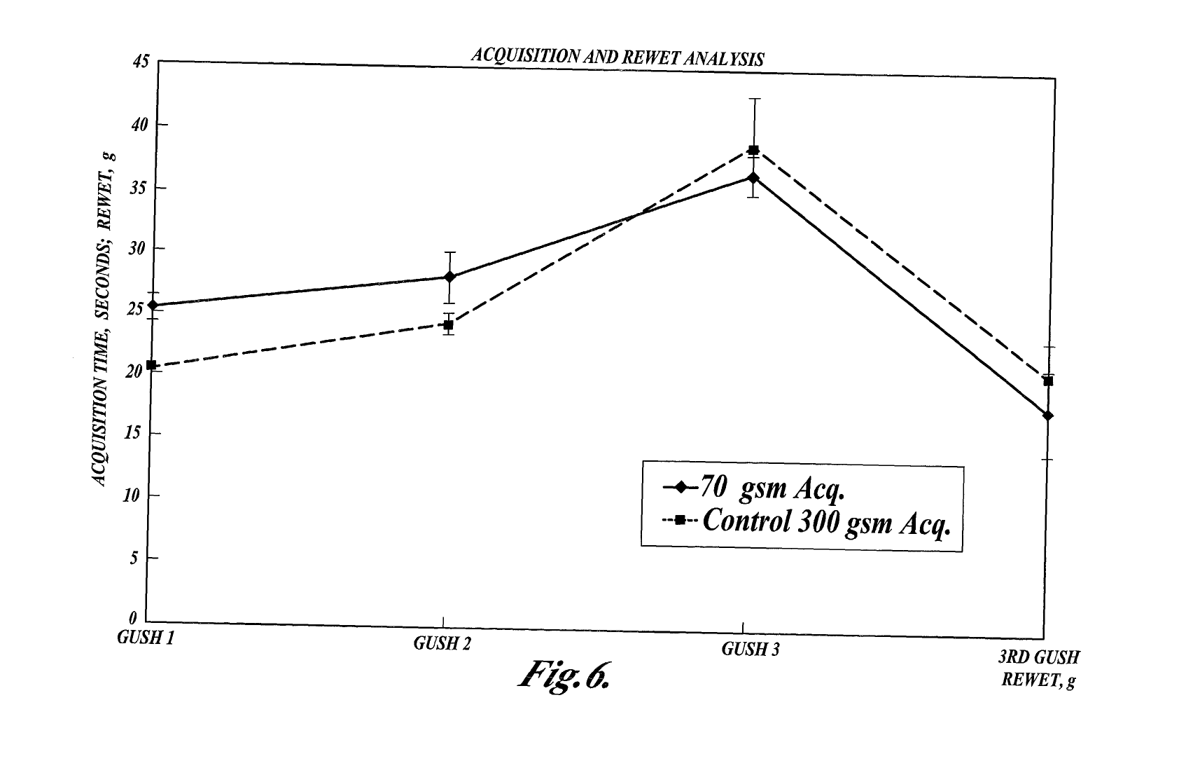

Referring to FIG. 5, another absorbent article includes a backing sheet 18, a storage layer 20, an intermediate layer 24, unitary absorbent layer 10, and a facing sheet 16. The intermediate layer can be incorporated into the article to increase the article's integrity or as a distribution layer to enhance the distribution of liquid from the acquisition layer to the storage layer. The intermediate layer 24 contains, for example, a densified fibrous material such as a combination of cellulose acetate and triacetin, which are combined just prior to forming the article. The intermediate layer 24 can thus bond to both the unitary absorbent layer 10 and the storage layer 20 to form an absorbent article having significantly more integrity than one in which the unitary absorbent layer and storage layer are not bonded to each other. The hydrophilicity of layer 24 can be adjusted in such a way as to create a hydrophilicity gradient among layers 10, 24, and 20. It should be understood that an independent intermediate layer is not required in order to get layer-to-layer bonding. When one of two adjacent layers or both layers contain a binder, if the two layers are brought together when the bonding medium is still active, bonding between the two layers will occur and provide a stronger composite compared to a composite lacking any bonding.

The unitary absorbent layer of the present invention improves the wet and dry integrity, surface dryness, rewet performance, and acquisition rate of absorbent products and articles that incorporate the absorbent layer. The unitary absorbent layer also provides increased pad integrity, improved appearance, and a reduction in wet collapse during use for absorbent products that incorporate the composite. Furthermore, because the unitary absorbent layer can be manufactured and delivered in web form, absorbent product manufacturing processes that include the absorbent layer are simplified relative to manufacturing processes that involve the handling of bales of crosslinked fibers or fluff pulp.

### EXAMPLES

The following examples are provided for the purposes of illustration, and not limitation.

### Example 1

### Unitary Absorbent Layer Formation: Laboratory Foam Method

This example illustrates a laboratory foam method for forming a representative unitary absorbent layer of the present invention. In this example, the absorbent acquisition is composed of 80% crosslinked cellulose fibers (Weyerhaeuser Co.) and 20% Celbond® T-105 (Hoechst Celanese).

Fiber Preparation

A lab size Waring blender was filled with 4L of water and Celbond® T-105 was added. The mixture was blended for short time to “open” the synthetic fibers. The crosslinked cellulose fibers were then added to the Celbond® T-105/water mixture and blended for at least one minute to “open” the crosslinked fibers and to effect mixing of the two fibers. The resulting aqueous mixture of fibers contained approximately 0.1% solids.

The crosslinked cellulose fiber-Celbond® T-105 mixture was placed in a container and water added to form an aqueous mixture having approximately 0.2% solids. The resulting mixture was then blended for a few seconds with an air-entrapping blade. A surfactant (Incronan 30, Croda, Inc.) was added to the blended mixture. Approximately 1 g active surfactant solids per gram fiber were added. The mixture was blended while slowly raising the mixer blade height with the rising foam. After about one minute, the mixing was terminated, and then restarted for another minute at constant mixer blade height. The resulting foam-fiber mixture has a volume of about three times the volume of the original water-fiber mixture.

Sheet Formation

The crosslinked cellulose fiber-Celbond® T-105 foam-fiber mixture was rapidly poured into a sheet mold having an inclined diffusion plate. After the addition of the foam-fiber mixture, the plate was removed from the mold, and a strong vacuum was applied to reduce the foam-fiber height. After the disappearance of most of the visible foam, the resulting sheet was removed from the mold and passed along with a forming wire over a slit couch to remove all excess foam and water.

The representative unitary absorbent layer was produced by placing the resulting damp sheet in a through air dryer to dry and to effect bonding.

### Example 2

### Unitary Absorbent Layer Formation: Commercial Foam Method

This example illustrates a commercial foam method for forming a representative unitary absorbent layer of the present invention. In this example, the unitary absorbent layer is composed of 80% crosslinked cellulose fibers (Weyerhaeuser Co.) and 20% Celbond® T-105 (Hoechst Celanese).

Fiber Preparation

Foam-fiber mixtures were prepared by combining dry fibers with surfactant and mixing for approximately 2 minutes with an air-entrapping blade. The crosslinked cellulose fiber-Celbond® fiber mixture was placed in a single tank.

Sheet Formation

Using positive displacement pumps, the foamy fiber slurry prepared as described above was pumped to an inclined multilayer headbox where the crosslinked cellulose fiber-Celbond® fiber mixture was laid down. The wire was passed over the slit couch vacuum.

The representative unitary absorbent layer was produced by placing the resulting damp sheet in a through air dryer to dry and to effect bonding.

### Example 3

### Method for the Evaluation of Acquisition Time and Rewet for Representative Unitary Absorbent Layers

The performance characteristics of representative unitary absorbent layers of the present invention were evaluated by incorporating the unitary absorbent layer into a commercially available diaper and comparing the acquisition time and rewet relative to a control diaper. The control diaper is a commercially available diaper that has been modified to include a crosslinked cellulose web having a basis weight of 300 g/m². The acquisition time and rewet were determined in accordance with the multiple-dose rewet test described below.

Briefly, the multiple-dose rewet test measures the amount of synthetic urine released from an absorbent structure after each of three liquid applications, and the time required for each of the three liquid doses to wick into the product.

A preweighed sample of the absorbent structure is prepared for the test by determining the center of the structure's core, measuring 1 inch to the front for liquid application location, and marking with “X” and then placing a liquid application funnel (minimum 100 mL capacity, 5-7 mL/s flow rate) 4 inches above surface of sample. Commercially available diapers are used as controls, and these diapers incorporating the unitary absorbent layer of the present invention were used for the comparative evaluation. Diapers incorporating the unitary absorbent layer were prepared by cutting and inserting the unitary absorbent layer into the diapers.

Once the sample is prepared, the test was conducted as follows. Flatten the sample, nonwoven side up, onto tabletop under the liquid application funnel. Fill funnel with dose (100 mL) of synthetic urine. Place dosing ring (5/32 inch stainless steel, 2 inch ID×3 inch height) onto the “X” measured on the sample. Apply first dose of synthetic urine within the dosing ring. Using a stopwatch, record the liquid acquisition time in seconds from the time the funnel valve is opened until the liquid wicks into the product from the bottom of the dosing ring. Wait twenty minutes. During the 20-minute waiting period after the first dose is applied, weigh a stack of filter papers (19-22 g, Whatman #3, 11.0 cm or equivalent, preexposed to room humidity for minimum of 2 hours before testing). During the second dose waiting period, take any dry filter papers left from first dose and add additional dry papers to total 29-32 g. During the third dose waiting period, take any dry papers and add additional dry papers to total 39-42 g. Place the stack of preweighed filter papers (i.e., dry blotter weight in Tables 1-9 below) on center of the wetted area and place cylindrical weight (8.9 cm diameter, 9.8 lb.) on top of these papers. Wait two minutes. Remove weight and weigh the papers. Record the weight change. Repeat the procedure two more times (i.e., for the second and third doses).

Rewet is reported as the amount of liquid absorbed back into the filter papers after each liquid dose (i.e., weight of wet filter papers−weight of dry filter papers).

Liquid acquisition time is reported as the length of time (seconds) necessary for the liquid to be absorbed into the product for each of the three doses.

The aqueous solution used in the tests is a synthetic urine available from National Scientific under the trade name RICCA. The synthetic urine is a saline solution containing 135 meq./l sodium, 8.6 meq./l calcium, 7.7 meq./l magnesium, 1.94% urea by weight (based on total weight), plus other ingredients.

Multiple-dose rewet test results for a control diaper and a diaper incorporating a representative unitary absorbent layer of the present invention are described in Example 4.

### Example 4

### Evaluation of Acquisition Time and Rewet for a Representative Unitary Absorbent Layer

This example compares the acquisition time and rewet performance of a wet laid acquisition layer and a representative unitary absorbent layer of the present invention. Multiple-dose rewet tests were performed as described above in Example 5 for a commercially available whole diaper (Proctor & Gamble) incorporating a wet laid fiber patch (80% crosslinked cellulose fibers and 20% Celbond® T-105, Hoechst Celanese) having a basis weight of 70 g/m² and a diaper incorporating a representative unitary absorbent layer (80% crosslinked cellulose fibers and 20% Celbond® T-105, Hoechst Celanese) produced by a process as generally described above in Example 1. The results are graphically illustrated in FIG. 6.

### Example 5

### Evaluation of Wet Tensile Strength for Representative Unitary Absorbent Layers

This example compares wet tensile strength of an air-laid acquisition composition to representative unitary absorbent layers of the present invention produced by wet-laid and foam-formed processes.

The wet tensile strength was determined for an air-laid 100% crosslinked cellulose fiber acquisition patch (basis weight 300 g/m²) and representative unitary absorbent layers (80% crosslinked cellulose fibers and 20% Celbond® T-105) produced by a foam-formed process. Representative foam-formed absorbent layers having basis weights of 40 and 50 g/m², prepared as generally described above in Example 2, were evaluated.

The tensile strength of the composites was measured by a horizontal tensile test method that measures the tensile and elongation properties of composites using a constant rate of elongation (CRE) machine that includes a horizontal jig apparatus affixed to a lower crosshead. The test method provides accurate measurements of breaking and stretching loads.

The composites to be tested were conditioned for at least 24 hours at 50% relative humidity and 23° C. After conditioning, composite specimens (10 cm×10 cm) were cut with a die cutter. The CRE machine was set up by affixing the horizontal jig to the lower crosshead with tightening. The upper jaws were then removed and a 25 kg load cell attached to the jig. The CRE controls were then set as follows: crosshead speed 25.4 mm/min.; chart speed 127 mm/min.; gauge length between clamps set to 50.8 mm. The CRE machine was then calibrated and sufficient air pressure (about 30 psi) provided to the clamps. Immediately prior to testing, the specimens were placed on a wire mesh and immersed in a liquid (synthetic urine) until saturated. The specimen was allowed to drain before being carefully placed onto the jig. The composite specimen was then clamped to the jig and the mesh removed. The CRE was then activated and the test continued until the specimen was torn.

The results demonstrate that foam-formed unitary absorbent layers had wet tensile strengths significantly greater than the air-laid composite. While the air-laid composite (basis weight 300 g/m²) had a wet tensile strength that was not measurable by the test method, the representative foam-formed unitary absorbent layers having basis weights of 40 and 50 g/m² had wet tensile strengths of about 90 and 120 g/inch, respectively.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. An absorbent layer comprising crosslinked cellulosic fibers and a binder, wherein the absorbent layer has a uniform porous structure.

2. The absorbent layer of claim 1 further comprising noncrosslinked fibers.

3. The absorbent layer of claim 1 further comprising synthetic fibers.

4. The absorbent layer of claim 3 wherein the synthetic fibers are selected from the group consisting of polyethylene terephthalate, polyethylene, polypropylene, nylon, and rayon fibers.

5. The absorbent layer of claim 3 wherein the synthetic fibers have a length up to about 2 inches.

6. The absorbent layer of claim 3 wherein the synthetic fibers have a length from about 0.25 to about 1.25 inches.

7. The absorbent layer of claim 3 wherein the synthetic fibers have a denier from about 5 to about 20.

8. The absorbent layer of claim 3 wherein the synthetic fibers are crimped fibers.

9. The absorbent layer of claim 1 wherein the binder is present in an amount from about 10 to about 30 percent by weight of the total composite.

10. The absorbent layer of claim 1 wherein the binder is selected from the group consisting of thermoplastic and thermosetting bonding agents.

11. The absorbent layer of claim 10 wherein the thermoplastic bonding agent is a multicomponent binding fiber.

12. The absorbent layer of claim 1 wherein the binder is a wet strength agent is selected from the group consisting of a polyamide-epichlorohydrin resin and a polyacrylamide resin.

13. The absorbent layer of claim 1 having a basis weight from about 40 to about 400 g/m².

14. The absorbent layer of claim 1 having a density from about 0.04 to about 0.10 g/m³.

15. The absorbent layer of claim 1 further comprising a superabsorbent polymeric material.

16. The absorbent layer of claim 1 having a basis weight of about 50 g/m² and a tensile strength of about 120 g/inch.

17. The absorbent layer of claim 1 having a basis weight of about 40 g/m² and a tensile strength of about 90 g/inch.

18. An absorbent layer comprising crosslinked cellulose fibers and multicomponent binding fibers, wherein the absorbent layer has a uniform porous structure.

19. The absorbent layer of claim 18 further comprising polyethylene terephthalate fibers.

20. The absorbent layer of claim 18 wherein the crosslinked fibers and binding fibers are thermally bonded.

21. An absorbent article comprising an absorbent layer comprising crosslinked cellulosic fibers and a binder, wherein the absorbent layer has a uniform porous structure.

22. An absorbent article comprising:
(a) a liquid pervious topsheet;
(b) an absorbent layer comprising crosslinked cellulosic fibers and a binder, wherein the absorbent layer has a uniform porous structure; and
(c) a liquid impervious backsheet.

23. An absorbent article comprising:
(a) a liquid pervious topsheet;
(b) an absorbent layer comprising crosslinked cellulosic fibers and a binder, wherein the absorbent layer has a uniform porous structure;
(c) a storage stratum comprising an absorbent fibrous material; and
(d) a liquid impervious backsheet.

24. An absorbent article comprising:
(a) a liquid pervious topsheet;
(b) an absorbent layer comprising crosslinked cellulosic fibers and a binder, wherein the absorbent layer has a uniform porous structure;
(c) a storage layer comprising an absorbent fibrous material;
(d) an intermediate layer interposed between the absorbent layer and the storage layer; and
(e) a liquid impervious backsheet.

25. The absorbent article of claim 24 wherein the intermediate layer comprises a liquid pervious tissue.

26. The absorbent article of claim 24 wherein the intermediate layer comprises a distribution layer.

27. The absorbent article of claim 26 wherein the distribution layer comprises hydrophilic fibers and a binder.

28. The absorbent article of claim 27 wherein the hydrophilic fibers comprise crosslinked cellulosic fibers.

29. The absorbent article of claim 26 wherein the distribution layer further comprises superabsorbent polymeric material.

30. The absorbent article of claim 22 wherein the article is a feminine care product.

31. The absorbent article of claim 23 wherein the article is a diaper.

32. The absorbent article of claim 25 wherein the article is an incontinence product.

33. The absorbent article of claim 26 wherein the article is a diaper.
